(19) United States
(12) Patent Application Publication
(10) Pub. No.: US 2021/0011043 A1
(43) Pub. Date: Jan. 14, 2021

(54) REAGENT DEPOSITION VERIFICATION

(71) Applicant: HEWLETT-PACKARD DEVELOPMENT COMPANY, L.P., SPRING, TX (US)

(72) Inventors: Suzanne Mirashrafi, Corvallis, OR (US); Christie Dudenhoefer, Corvallis, OR (US); Heather B. Louderback, Corvallis, OR (US); Craig Olbrich, Corvallis, OR (US); Dennis R. Esterberg, Corvallis, OR (US); Jeffrey A. Nielsen, Corvallis, OR (US)

(73) Assignee: HEWLETT-PACKARD DEVELOPMENT COMPANY, L.P., SPRING, TX (US)

(21) Appl. No.: 16/500,362

(22) PCT Filed: Jul. 18, 2017
(86) PCT No.: PCT/US2017/042534

§ 371 Date: Oct. 2, 2019

(51) Int. Cl. G01N 35/10; G01N 35/00; G01N 35/02

(52) U.S. Cl. CPC G01N 35/1002; G01N 35/1009; G01N 35/02; G01N 35/00732; G01N 35/1065

(57) ABSTRACT

A reagent substrate may include a reagent sample deposition area on which a sample of a reagent is deposited, and a number of diagnostic regions on which a diagnostic sample of the reagent is deposited for verification of deposition of the diagnostic sample. A reagent dispensing system may include a conveyor surface to convey a number of substrates, at least one reagent module located in-line with respect to the conveyor surface where the reagent module includes at least one reagent dispensing device to dispense a reagent on the substrates, and at least one optical sensor to verify the dispensing of the reagent at a number of diagnostic regions of the substrate.

### BACKGROUND

The life sciences research and associated diagnostic industries use a number of reagents and patient samples to perform testing and diagnostics. Dispensing liquids such as these reagents and patient samples in quantities from picoliters to microliters may be used in many areas of pharmaceutical and biology research. For example, dispensing a number of reagents in these quantities may be useful in medical and veterinary diagnostics, forensics testing, and agricultural testing to determine the presence of a chemical or biological in a sample. Even within these fields, low-volume liquid dispensing may be used for many different operations.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a reagent dispensing system, according to an example of the principles described herein.

FIG. 2 is a block diagram of a substrate onto which a number of reagents is dispensed, according to an example of the principles described herein.

FIG. 3 is a block diagram of a computer program product for on-substrate verification of reagent deposition, according to an example of the principles described herein.

FIG. 4 is a block diagram of a reagent dispensing system, according to another example of the principles described herein.

FIG. 5 is a block diagram of a reagent module, according to an example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Human interaction during life science research and diagnostic processes may lead to mistakes in those processes. Such mistakes may decrease the likelihood of scientific breakthroughs and increase the likelihood of misdiagnosis of patients illnesses. Further, with human interaction, these processes may prove tedious thereby increasing the costs associated with these processes as well as increase the time spent completing the processes. Automation of these processes, however, limits mistakes, time, and costs.

Instruments and tools used in life science research and diagnostic processes have been developed to increase efficiency, decrease costs, and decrease time spent conducting this research or completing diagnosis. However, even with these developments, increased numbers of reagents used to interact with a sample increase the complexity and time of completing those tasks.

In an automated, computer-driven diagnostics system, reagents may be dispensed based on a number of test protocols, and a wide variety and volumes of different reagents may be dispensed based on these test protocols. However, in some systems, the dispensing device may dispense reagents errantly due to the abilities of the dispensing device to dispense the reagent. Further, some reagent dispensing devices may become clogged or obstructed due to dispensing a reagent that tends to encourage such obstructing. This clogging issue may be exacerbated if the reagent dispersion system dispenses a wide range of reagents with different properties that may each obstruct the reagent dispensing devices in a different way or in different degrees.

The reagents may be dispensed onto a substrate such as, for example, microscope slides containing biological samples for research or medical diagnoses using the reagent dispensing devices, and the accuracy and reliability of the dispensing may be verified. For example, a reagent dispensing device may inaccurately deposit a reagent on the substrate such that the reagent does not contact a biological sample as may be intended. In this situation, the testing may prove faulty or provide a false positive resulting in an inaccurate test. This inaccurate deposition may even go undetected in situations where the reagent is clear and does not tint an untreated surface or otherwise cannot be detected by a technician. Thus, a verification system that verifies that a reagent is deposited as a technician has desired or intended, may greatly reduce or eliminate mistakes that may occur in a reagent deposition system.

Examples described herein provide a computer program product for on-substrate verification of reagent deposition. The computer program product may include a computer readable storage medium including computer usable program code embodied therewith. The computer usable program code, when executed by a processor identifies a sample region of a substrate, identifies at least one diagnostic region of the substrate, deposits, with a reagent dispensing devices, a reagent on the at least one diagnostic region, and verifies deposition of the reagent at the at least one diagnostic region of the substrate.

The computer program product may include computer usable program code to, when executed by a processor, pretreat the substrate, the pretreating facilitating the verification of the deposition of the reagent at the diagnostic region. Further, the computer program product may include computer usable program code to, when executed by a processor, instruct at least one optical sensor to verify the deposition of the reagent at the at least one diagnostic region.

Identifying at least one diagnostic region of the substrate may include identifying a first diagnostic region on the substrate before a sample deposition area with respect to a direction of travel of the substrate, identifying a second diagnostic region on the substrate after a sample deposition area with respect to the direction of travel of the substrate, or combinations thereof. Pretreating the substrate may include etching the at least one diagnostic region, pH treating the at least one diagnostic region, applying a label to the at least one diagnostic region, or combinations thereof.

The computer program product may include computer usable program code to, when executed by a processor, deposit a reagent on the at least one diagnostic region in a pattern with a number of reagent dispensing devices. The pattern identifies a health of the reagent dispensing device, identifies a clogging of a number of nozzles of the reagent dispensing device, comprises a volume gradient of the reagent, comprises a concentration gradient of the reagent, comprises a barcode identifying the substrate, comprises a barcode identifying a sample test protocol, or combinations thereof.

The computer program product may include computer usable program code to, when executed by a processor, service the reagent dispensing device in response to the verification of the deposition of the reagent at the at least one diagnostic region of the substrate. Further, the computer program product may include computer usable program code to, when executed by a processor, change a nozzle mask in response to a determination that a concentration of the reagent deposit on the at least one diagnostic region is incorrect. Still further, the computer program product may include computer usable program code to, when executed by a processor, mark the substrate as faulty in response to a determination that the deposition of the reagent at the at least one diagnostic region of the substrate was compromised.

Examples described herein provide a reagent dispensing system. The reagent dispensing system may include a conveyor surface to convey a number of substrates, at least one reagent module located in-line with respect to the conveyor surface where the reagent module comprising at least one reagent dispensing device to dispense a reagent on the substrates, and at least one optical sensor to verify the dispensing of the reagent at a number of diagnostic regions of the substrate. The reagent dispensing device may be a digitally addressable fluid ejection device. The at least one optical sensor may include two optical sensors. The two optical sensors may include a first optical sensor to image the substrate previous to the dispensing of the reagent on the substrate, and a second optical sensor to verify dispensing of the reagent after a dispensing operation performed by the reagent module.

Examples described herein provide a reagent substrate. The reagent substrate may include a reagent sample deposition area on which a sample of a reagent is deposited, and a number of diagnostic regions on which a diagnostic sample of the reagent is deposited for verification of deposition of the diagnostic sample. The diagnostic regions of the substrate may include untreated portions, etched portions, pH treated portions, labeled portions, or combinations thereof.

As used in the present specification and in the appended claims, the term “a number of” or similar language is meant to be understood broadly as any positive number comprising 1 to infinity; zero not being a number, but the absence of a number.

In the following description, for purposes of explanation, numerous specific details are set forth in order to provide a thorough understanding of the present systems and methods. It will be apparent, however, to one skilled in the art that the present apparatus, systems, and methods may be practiced without these specific details. Reference in the specification to “an example” or similar language means that a particular feature, structure, or characteristic described in connection with that example is included as described, but may or may not be included in other examples.

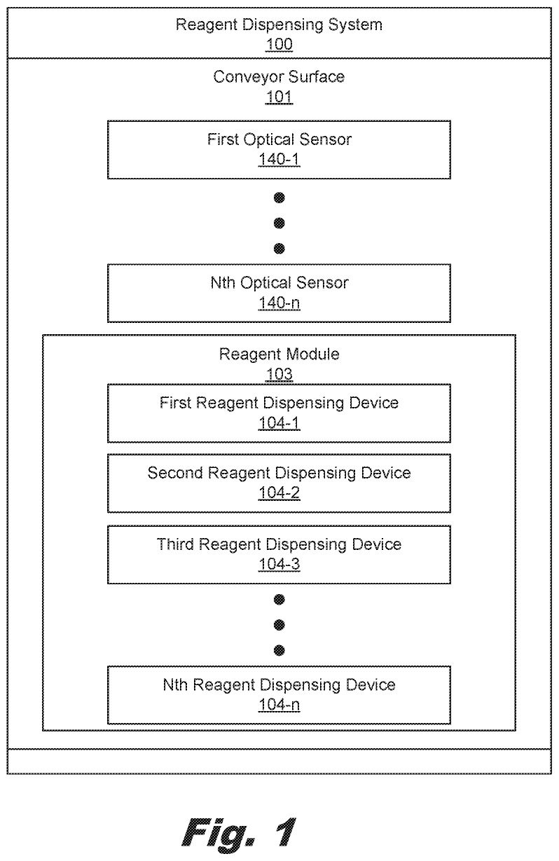

Turning now to the figures, FIG. 1 is a block diagram of a reagent dispensing system (100), according to an example of the principles described herein. The reagent dispensing system (100) may include a substrate conveyance system (101) on which a number of substrates are conveyed underneath a reagent module (103) of the reagent dispensing system (100). The direction of travel of the substrate conveyance system (101) is indicated by arrows (470). In one example, the substrates may be microscope slides, test samples, cell-culture dishes such as Petri dishes, paper, tissue samples, porous media, lateral flow strip media, coated media, microtiter plates, a conveyance web, or other substrates. The substrate conveyance system (101) may be, for example, a conveyor belt that moves a number of substrates under an area where the reagent module (103) may deposit a number of reagents onto the substrate. The substrate conveyance system may travel in either a positive x-direction or a negative x-direction in order to allow the substrates to move left to right or right to left under the reagent module (103). This allows the substrates to have a plurality of reagents deposited on them, and may also allow for chemical reactions to occur on the substrate before a subsequent reagent is deposited on the substrate. However, any other type of conveyance system may be used such as, for example, mechanical stages that moves in and out of the reagent deposition area (402), rolls or reels of flexible material that moves underneath the reagent dispensing devices (104) within the reagent module (103), or other conveyance systems.

The reagent module (103) may include a module frame to mechanically and electrically couple the reagent dispensing devices (104) within the reagent module (103) to the reagent dispensing system (100). The module frame may include a number of mechanical interfaces to align the reagent dispensing devices (104) with respect to the reagent module (103). Further, the module frame may include a number of electrical interfaces to electrically couple the reagent dispensing devices (104) to the reagent module (103), and, in turn, the reagent dispensing system (100). Signals may be sent by the reagent dispensing system (100) to the reagent dispensing devices (104) via the number of electrical interfaces of the reagent module (103). These signals may be used to instruct the reagent dispensing devices (104) to dispense a volume of reagent onto a substrate located on the substrate conveyance system (101).

Each of the reagent dispensing devices (104) may be any device that dispenses a number of reagents. In one example, the reagent dispensing devices (104) may include devices that dispense different volumes of reagents. For example, a first reagent dispensing device (104-1) may dispense a first range of volumes of a reagent, a second reagent dispensing device (104-2) may dispense a second range of volumes of a reagent where the second range of volumes may be more voluminous relative to the first range of volumes, and a third reagent dispensing device (104-3) may dispense a third range of volumes of a third reagent where the third range of volumes may be more voluminous relative to the second range of volumes.

As depicted using the ellipses in FIG. 1 and the inclusion of an Nth reagent dispensing device (104-n), any number of reagent dispensing devices may be included within a reagent module (103). Each of these reagent dispensing devices (104-1, 104-2, 104-3, 104-n, collectively referred to herein as 104) may have differing architectures or form factors that allow them to dispense their respective volumes. For example, the first reagent dispensing device (104-1) may include a cassette device that dispenses between approximately 1 picoliter (pL) and 10 microliters (μL) as the first range of volumes of a reagent. The cassette may include, for example, a T8+ or D4+ dispensehead cassette produced and distributed by HP, Inc. With these types of cassettes, a relatively small amount of reagent may be dispensed at a given time. In one example, the first reagent dispensing device (104-1) or cassette may be any digitally addressable fluid ejection device that contains a very small amount of reagent on the order of approximately up to 1 milliliter (mL). Further, the fluid within the first reagent dispensing device (104-1) may come pre-filled in the first reagent dispensing device (104-1), or a technician may fill the first reagent dispensing device (104-1) with a pipette or similar preliminary, manual reagent dispensing device at the time of use of the first reagent dispensing device (104-1).

Further, in one example, the second reagent dispensing device (104-2) may include a digitally addressable fluid ejection device. In this example, the digitally addressable fluid ejection device may include a number of fluid ejection die to dispense the second range of volumes of a reagent. For example, the second reagent dispensing device (104-2) may include a digitally addressable fluid ejection device that dispenses between approximately 100 nanoliters (nL) and 100 μL as the second range of volumes of a reagent. In one example, the digitally addressable fluid ejection device may be a thermal or piezoelectric fluid ejection device where the reagents are dispensed from an array of fluid ejection chambers and nozzles of the fluid ejection die using thermal expansion or piezoelectric forces applied to the reagents. In this example, the second reagent dispensing device (104-2) may contain, for example, 1 to 40 milliliters of reagent and may be pre-packaged with the reagent before the time of use.

The third reagent dispensing device (104-3) may include a digitally addressable fluid ejection device that dispenses between approximately 100 nanoliters (nL) and 100 μL as the third range of volumes of a reagent. In this example, the third reagent dispensing device (104-3) may contain bulk volumes of reagent since this bulk reagent dispensing device may be used most often, Thus, third reagent dispensing device (104-3) may contain for example, 40 to 1,000 mL of reagent and may be pre-packaged with the reagent before the time of use. In one example, the third reagent dispensing device (104-3) may include any high-volume reagent dispensing device such as, for example, a digitally addressable fluid ejection device fluidically coupled to an off-line bulk supply of reagent. In this example, the third reagent dispensing device (104-3) may be used in connection with the dispensing of bulk amounts of reagents onto the substrate.

The reagent dispensing system (100) may further include a number of optical sensors (140-1, 140-n, collectively referred to herein as 140). The optical sensors (140) may be any type of sensor capable of detecting deposition of a reagent on a substrate. In one example, the optical sensors (140) may detect the existence, the composition, the volume, the color, the temperature, a pattern, other properties of the dispensed reagent, or combinations thereof. In one example, a first optical sensor (140-1) may be used to image a substrate before a reagent is deposited thereon, and a second optical sensor (140-n) may be used to image the substrate after a reagent has been deposited thereon. In this example, the two optical sensors (140) may work in tandem to confirm the properties of the dispensed reagent. More regarding the reagent dispensing system (100) is described herein. Further, in one example, the optical sensors (140) may be located anywhere in the reagent dispensing system (100) where the optical sensors (140) may view the dispensing of the reagent or the reagent as dispensed on the substrate. For example, the optical sensors (140) may be located on edges of well plates, above slides on the conveyance system (101), or from some other vantage point from which the dispensing of the reagent may be viewed or from which the dispensed reagent on the substrate (200) may be viewed. Thus, in some examples, the optical sensors (140) may view the deposition of reagents in situ.

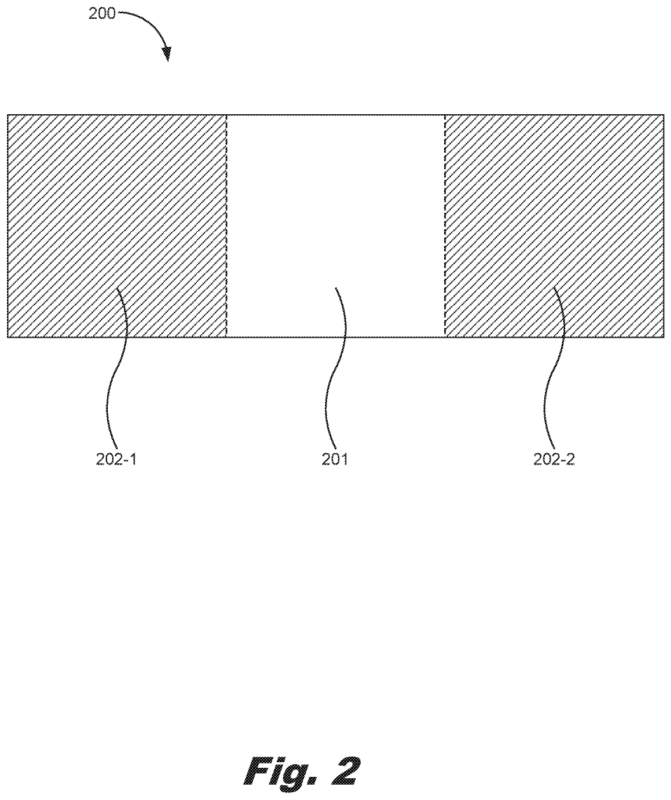

FIG. 2 is a block diagram of a substrate (200) onto which a number of reagents is dispensed, according to an example of the principles described herein. The substrate (200) may be any object on which a reagent may be dispensed, and, in one example, may include microscope slides, test samples, cell-culture dishes such as Petri dishes, paper, tissue samples, or other substrates.

The substrate (200) may include a sample region (201) onto which a number of reagents may be deposited by the reagent dispensing devices (104) of the reagent module (103) to effectuate a diagnostic process according to, for example, a desired test protocol administered by a technician through the use of the reagent dispensing system (100). In one example, a plurality of reagents may be dispensed onto the sample region (201) of the substrate (200). In this example, the substrate (200) may be exposed to the reagent module (103) and its reagent dispensing devices (104) to receive the plurality of reagents. The substrate conveyance system (101) may move a single substrate into a position under the reagent dispensing devices (104) any number of times in order to receive the plurality of reagents on the sample region (201) of the substrate (200). In one example, a plurality of samples may be deposited within the sample region (201) between the diagnostic regions (202). In this example, the plurality of samples may be part of separate reactions.

The substrate (200) may also include a number of diagnostic regions (202-1, 202-2). The diagnostic regions (202-1, 202-2) of the substrate (200) are used to verify a reliable and effectual dispensing of the reagents, which is indicative of good health or correct functioning of the reagent dispensing devices (104) in dispensing their respective reagents. The verification of reliable dispensing and good nozzle health will be done directly on the substrates (200) in regions that do not interfere with the area containing the immobilized sample. With respect to a direction of travel of the substrate conveyance system (101), the number of diagnostic regions (202-1, 202-2) may be located before or after the sample region (201), or may, in another example, before and after the sample region (201).

In an example where the first diagnostic region (202-1) is located before the sample region (201) relative to the direction of travel of the substrate conveyance system (101), the optical sensors (140) may image the first diagnostic regions (202-1) before and after deposition of a reagent to determine whether the reagent dispensing devices (104) are healthy and ready to print before dispensing a potentially valuable or scarce reagent onto the sample region (201). In an example where the second diagnostic region (202-2) is located after the sample region (201) relative to the direction of travel of the substrate conveyance system (101), a defective or ineffective dispensing of the reagents may be detected based on the supposition that if the dispensed reagent was defectively or ineffectively dispensed on the second diagnostic region (202-2), that dispensing of the reagent on the sample region (201) was also defective or ineffective.

In an example where both the first diagnostic region (202-1) and the second diagnostic region (202-2) are utilized, the dispensing of the reagents may show the state of the reagent dispensing devices (104) before and after the reagents are dispensed on the sample region (201) of the substrate (200), which may change while dispensing the reagents on the sample region (201). Thus, by comparing the deposition of the reagents on the first diagnostic region (202-1) to the deposition of the reagents on the second diagnostic region (202-2), any defective or ineffective dispensing of the reagents may be detected. More regarding the deposition of reagents by the reagent dispensing system (100) is described herein.

In one example, the diagnostic regions (202-1, 202-2) may be located on a separate substrate (200) from a substrate (200) on which the sample region (201) is located. In this example, the reagents that are assigned to be dispensed in the diagnostic regions (202-1, 202-2) may be dispensed on their own substrates (200) among a number of substrates (200) presented on the conveyor surface (101). The optical sensors (140) may be used to image a substrate (200) on which a sample has been dispensed and a separate substrate (200) on which the diagnostics have been performed. In one example, a separate diagnostic substrate (200) on which diagnostics are performed may be placed between a plurality of sample substrates (200) on which samples are dispensed. In one example, the separate diagnostic substrates (200) may be presented between the sample substrates (200) at a predefined frequency allowing at least one separate sample substrate (200) to be presented between each separate diagnostic substrate (200).

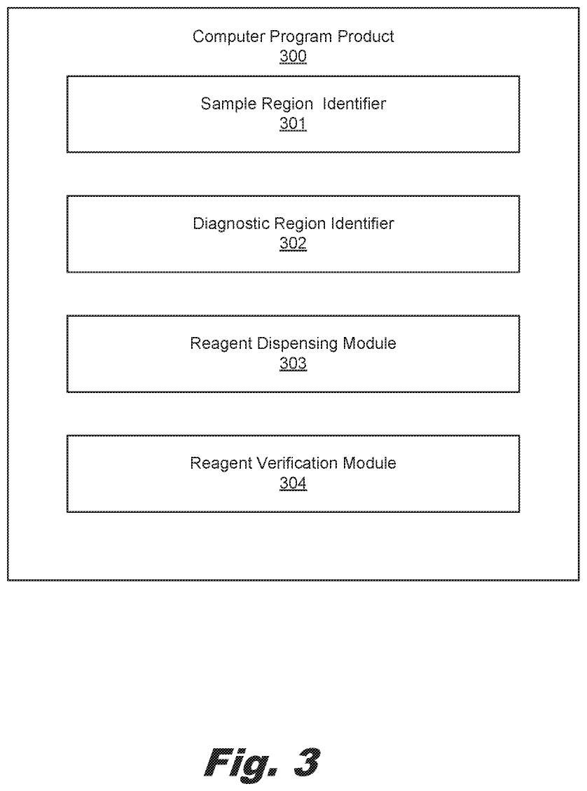

FIG. 3 is a block diagram of a computer program product (300) for on-substrate verification of reagent deposition, according to an example of the principles described herein. The computer program product (300) may include a computer readable storage medium comprising computer usable program code embodied therewith. The computer usable program code may, when executed by a processor of the reagent dispensing system (100), identify, with a sample region identifier (301) and through the use of the optical sensors (140), a sample region (201) of a substrate (200) before or after the reagents are deposited in the sample region (201) of a substrate (200), or both before or after the reagents are deposited in the sample region (201) of a substrate (200).

Further, the computer usable program code may, when executed by the processor of the reagent dispensing system (100), identify, with a diagnostic region identifier (302) and through the use of the optical sensors (140), at least one diagnostic region (201-1, 201-2) of the substrate (200) before or after the reagents are deposited in the diagnostic region (202-1, 202-2) of the substrate (200), or both before or after the reagents are deposited in the diagnostic region (202-1, 202-2) of the substrate (200). In one example, the diagnostic region identifier (302) identifies both the first diagnostic region (202-1) and the second diagnostic region (202-2).

With at least one reagent dispensing device (104), the computer usable program code may, when executed by the processor of the reagent dispensing system (100), instruct the reagent dispensing device (104) to deposit a reagent on the at least one diagnostic region (202-1, 202-2) in order to determine if the reagent dispensing devices (104) are defective or are otherwise ineffectively dispensing their respective reagents. In dispensing the reagent, the processor of the reagent dispensing system (100) may execute a reagent dispensing module (303) used to instruct a number of the reagent dispensing devices (104) to dispense their respective reagents.

Still further, the computer usable program code may, when executed by the processor of the reagent dispensing system (100), verify, with a reagent verification module (304) and through the use of the processing device, the deposition of at least one reagent on a diagnostic region (201-1, 201-2) of the substrate (200). In one example, the processor may compare the two images captured before and after the reagent dispensing module (303) is executed to verify whether the dispensed reagents were properly and effectively dispensed on the substrate (200).

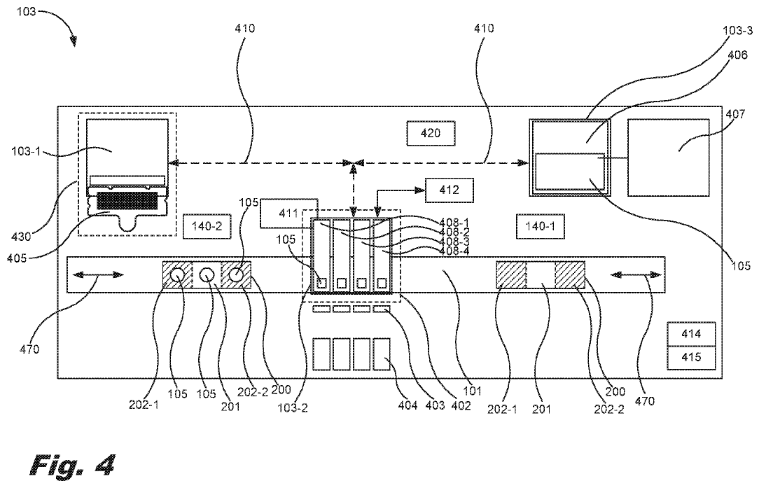

With reference to both FIGS. 2 and 3, the computer program product may also include additional computer usable program code to provide additional functionality to the reagent dispensing system (100). FIG. 4 is a block diagram of a reagent dispensing system (400), according to another example of the principles described herein. The reagent dispensing system (400) may include a number of elements including the reagent dispensing system (100) and its various elements described in connection with FIGS. 1 through 3. Therefore, similarly-numbered elements included in FIGS. 1 through 3 and described in connection with FIGS. 1 through 3 designate similar elements within the reagent dispensing system (400) of FIG. 4. As depicted in FIG. 4, a reagent module (103) is located within a reagent deposition area (402) of the reagent dispensing system (400) and is considered to be in an in-line position to dispense reagent (105) onto a number of substrates (200).

In one example, the reagent dispensing system (400) may include a wiping station (403) to wipe off or clean the reagent dispensing devices (104) of the reagent module (103). In one example, the wiping station (403) wipes a nozzle plate or other ejection surface of the at least one reagent dispensing device (104) within the reagent module (103). The reagent dispensing system (400) may also include a capping station (404) to seal a number of nozzles or other ejection devices of the at least one reagent dispensing device (104) from ambient atmosphere around the reagent deposition area (402).

The reagent dispensing system (400) of FIG. 4 includes a number of different reagent modules (103-1, 103-2, 103-3, collectively referred to herein as 103) that include differing architectures or form factors that allow them to dispense their respective volumes. For example, the in-line reagent module (103) located within the reagent deposition area (402) includes four digitally addressable fluid ejection devices (408-1, 408-2, 408-3, 408-4, collectively referred to herein as 408). In one example, the four digitally addressable fluid ejection devices (408) may each include a volume of reagent (105). The reagent module (103-2) including the digitally addressable fluid ejection devices (408) may dispense an average or mid-range amount of reagent (105), and may be used for dispensing reagents that are dispensed at an average or mid-range frequency.

Other reagent modules (103-1, 103-2, 103-3) may be stored or placed off-line, and may be exchangeable with the in-line reagent module (103-2). Other architectures or form factors of reagent modules (103) may be included within the reagent dispensing system (400). Another architecture or form factor of reagent modules (103) may include a reagent module (103-1) that includes a cassette device (405). The cassette device (405) may dispense between approximately 0.1 picoliters (pL) and 0.1 microliters (μL) as the first range of volumes of a reagent. The cassette device (405) may include, for example, a T8+ or D4+ dispensing cassette produced and distributed by HP, Inc. With these types of cassettes, a relatively small amount of reagent may be dispensed at a given time using a dispensing die that is capable of dispensing these relatively small volumes of fluid. The cassette device (405) may be used to dispense volumes of fluid that are less frequently dispensed relative to other reagents, are negatively susceptible to environmental conditions, are expensive to inventory, are mixed immediately before use, have a relatively short shelf life, have other properties that lend their use to relatively smaller volumes, or combinations thereof.

Another architecture or form factor of reagent modules (103) may include a reagent module (103-3) that includes a bulk reagent dispensing device (406). The bulk reagent dispensing device (406) may be fluidically coupled to a bulk reagent source (407) to provide the bulk reagent dispensing device (406) with ample reagent to dispense. The reagent module (103-3) including the bulk reagent dispensing device (406) may dispense a bulk or high-range amount of reagent (105), and may be used for dispensing reagents that are dispensed at above average frequencies.

As depicted in FIG. 4, a number of exchange paths (410) depicted as dashed lines indicate a path on which an off-line reagent module (103) may take when being switched with an in-line module (103). In this example, an automated storage and retrieval system (ASRS) (420) may be used to transport the reagent modules (103) to and from the reagent deposition area (402). The ASRS (420) may include any number of conveyor systems, lift systems, robotic arms, other ASRS systems, and combinations thereof. In another example, a technician may manually exchange the reagent modules (103), In another example, the in-line reagent module (103-2) may be exchanged for an off-line reagent module (103-1, 103-3) by a user such as a technician manually exchanging the reagent modules (103).

The reagent dispensing system (400) may further include at least one environmentally-controlled area (430) to preserve a number of reagents (105) within the reagent modules (103). Many reagents dispensable by the reagent modules (103) may have a shelf life or may perform better in a reaction if they are stored in an area where its environment may be controlled. The environmentally-controlled areas (430) may be environmentally sealed from the remainder of the reagent dispensing system (400), and may control, for example, a humidity level, a temperature, a pressure, or other environmental states within the reagent dispensing system (400).

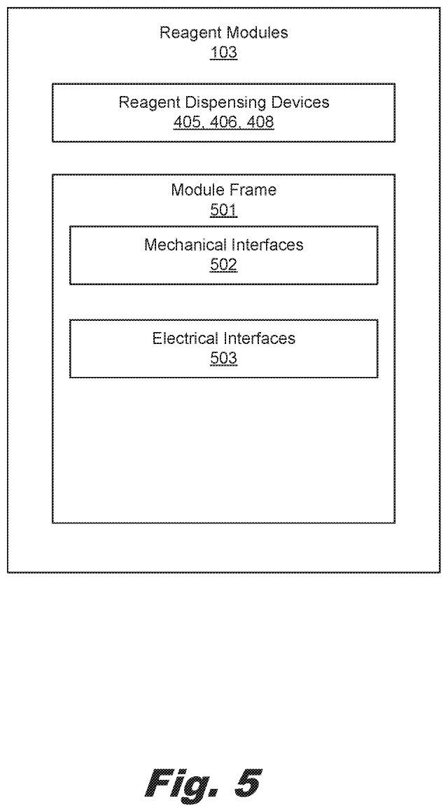

With continued reference to FIG. 4, FIG. 5 is a block diagram of a reagent module (103), according to an example of the principles described herein. When a reagent module (103) is placed in the reagent deposition area (402), a number of mechanical (411) and electrical (412) interfaces within the reagent dispensing system (400) and located at the reagent deposition area (402) interface with mating mechanical (502) and electrical (503) interfaces of the module frame (501) on the reagent modules (103). In this manner, the reagent modules (103) may be physically coupled to the reagent dispensing system (400) and be seated within the reagent deposition area (402) in order to dispense reagent (105) onto the substrates (200).

In one example, the module frame (501) is included within the reagent module (103). The module frame (501) mechanically and electrically couples the reagent dispensing devices (405, 406, 408) within the reagent module (103) to the reagent dispensing system (100). The module frame (501) may include the mechanical interfaces (502) to align the reagent dispensing devices (104) with respect to the reagent module (103). Further, the module frame may include the electrical interfaces (503) to electrically couple the reagent dispensing devices (405, 406, 408) to the reagent module (103), and, in turn, the reagent dispensing system (100). Signals may be sent by the reagent dispensing system (100) to the reagent dispensing devices (405, 406, 408) via the number of electrical interfaces (503) of the reagent module (103). These signals may be used to instruct the reagent dispensing devices (405, 406, 408) to disperse a volume of reagent (105) onto a substrate (450) located on the dispersion surface (101). In this manner, the reagent modules (103) may be physically coupled to the reagent dispensing system (400) and be seated within the reagent deposition area (402) in order to dispense reagent (105) onto the substrates (450).

Further, in this physically coupled state, a number of electrical interfaces (503) located on the reagent modules (103) electrically interface with the electrical interfaces (412) located in the reagent deposition area (402) of the reagent dispensing system (400). This allows the reagent dispensing system (400) to send instructions in the form of signals to the modules (103) that cause the various reagent dispensing devices (405, 406, 408) to dispense their respective reagents (105) onto the substrates (200).

A processing device (414) and a data storage device (415) may be included in the reagent dispensing system (400) to instruct and store data about the reagent modules (103) and their respective reagent dispensing devices (405, 406, 408). The processing device (414) may provide signals to the reagent dispensing devices (405, 406, 408) to instruct the reagent dispensing devices (405, 406, 408) to dispense their respective reagents (105) onto the substrates (200). Further, the processing device (414) may instruct the ASRS (420) to exchange the in-line reagent module (103) with an off-line reagent module (103) in order to dispense a different reagent or volume of reagent. The processing device (414) may further provide instructions to the conveyor surface (101) as to speed and direction in moving the substrates (200) within the reagent deposition area (402) and under the reagent modules (103). The processing device (414) may further actuate a number of optical sensors (140) to cause the optical sensors to detect dispensing of reagents onto the substrates (200) as described herein and receive data from the optical sensors (140).

The data storage device (415) may include various types of memory modules, including volatile and nonvolatile memory. For example, the data storage device (415) of the present example may include Random Access Memory (RAM), Read Only Memory (ROM), and Hard Disk Drive (HDD) memory. Many other types of memory may also be utilized, and the present specification contemplates the use of many varying type(s) of memory in the data storage device (415) as may suit a particular application of the principles described herein. In certain examples, different types of memory in the data storage device (415) may be used for different data storage needs. For example, in certain examples the processor (414) may boot from Read Only Memory (ROM), maintain nonvolatile storage in the Hard Disk Drive (HDD) memory, and execute program code stored in Random Access Memory (RAM). The data storage device (415) may comprise a computer readable medium, a computer readable storage medium, or a non-transitory computer readable medium, among others. For example, the data storage device (415) may be, but not limited to, an electronic, magnetic, optical, electromagnetic, infrared, or semiconductor system, apparatus, or device, or any suitable combination of the foregoing. More specific examples of the computer readable storage medium may include, for example, the following: an electrical connection having a number of wires, a portable computer diskette, a hard disk, a random-access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain, or store computer usable program code for use by or in connection with an instruction execution system, apparatus, or device. In another example, a computer readable storage medium may be any non-transitory medium that can contain, or store a program for use by or in connection with an instruction execution system, apparatus, or device. The data storage device (415) may store computer usable program code described herein for execution by the processor.

As depicted in FIG. 4, the conveyor surface (101) may move in a positive x-direction or a negative x-direction to move the substrates (200) underneath the reagent modules (103) in order to dispense reagent (105) onto the substrates (200). In doing so, the right substrate (200) depicted in FIG. 4 does not include a dispensed reagent (105), but the left substrate does include a dispense reagent (105). In one example, a number of reagents are depicted in FIG. 4 as having been dispensed on the sample region (201) and the diagnostic regions (202-1, 202-2) of the substrate (200). In this example, a first optical sensor (140-1) may capture an image of the substrate (200) before a number of reagents are dispensed onto the substrate (200). A second optical sensor (140-2) may capture an image of the substrate (200) after a number of reagents are dispensed onto the substrate (200).

The images captured by the optical sensors (140-1, 140-2) may be stored in the data storage device (415) and processed by the processing device (414) to determine or confirm the existence, the composition, the volume, the color, the temperature, a pattern, other properties of the dispensed reagent, or combinations thereof on the substrate (200) by analyzing the diagnostic regions (202-1, 202-2) of the substrate (200) within the images. In do so, the reagent dispensing system (400) may execute, with the processor (414), the reagent verification module (304). The diagnostic regions (202) of the substrate (200) within the images captured by the optical sensors (140) may be compared to determine whether the reagent was deposited on the substrate (200) in an intended or instructed manner.

In one example, the substrate may be pretreated. In many instances, the reagents (105) are clear, making imaging and identifying the reagent or the dispensed pattern of the reagent difficult for the optical sensors (140). Thus, some form of indicator may be added to the reagent such as a dye to make the reagent visible. In this example, the substrate (200) may be untreated or pretreated. However, in situations where adding a dye to the reagent may compromise the reagent, a pretreatment may be added to the substrate (200). In one example, pretreatment may include etchings in the substrate. In this example, the diagnostic regions (202-1, 202-2), the sample region (201), or both may be etched to allow for the reagent dispensing devices (104) to be used for labeling of the substrate (200). Labeling the substrate (200) may occur using the precision deposition provided by the reagent dispensing devices (104). The labeling may indicate a number assigned to the diagnostic test being run, the reagents (105) used in the diagnostic test, a substrate (200) number, whether the deposition of reagent (105) on the substrate (200) was performed correctly, other information, or combinations thereof.

As to the labeling indicating whether the deposition of the reagent (105) on the substrate (200) was performed correctly, the first optical sensor (140-1) may image the substrate (200) before deposition of the reagent (105), the deposition may occur on the first diagnostic region (202-1) and/or the sample region (201), and, in response to the second optical sensor (140-2) capturing an image of the substrate (200) and the processor (414) determining that the deposition of the reagent (105) was performed incorrectly or ineffectively, the processor (414) may instruct the reagent dispensing devices (104) to dispense the reagent (105) onto the second diagnostic region (202-2) in a pattern or in a textual manner that indicates that the reagent (105) was deposited on the substrate (200) incorrectly or ineffectively.

Another form of pretreating of the substrate (200) may include treating the substrate (200) with a pH reactive film. In this example, the pH reactive film may be chemically reactive to the presence of a reagent (105) such that it changes color or causes the reagent (105) to change color when exposed to reagents (105) of different pH levels. Many reagents (105) have identified pH levels, and use of this knowledge may assist in identifying the correctness and effectiveness of the deposition of the reagents (105) onto the substrate (200). In one example, the pretreatment of the substrate (200) may be performed previous to the substrate (200) being introduced to the reagent dispensing system (400). In another example, the substrate (200) may be pretreated with the pH reactive film using a number of the reagent dispensing devices (104) themselves that may contain the fluids used to form the pH reactive film on the substrate (200). In this example, the substrates (200) may be presented to the reagent module (103) for pretreatment, and a number of reagent dispensing devices (104) may dispense pH reactive fluids onto at least a portion of the substrate (200). In one example, the conveyor surface (101) may be a continuous conveyance web where the reagent is dispensed directly onto the conveyance web, as mentioned above. In this example, the conveyance web, serving as the substrate, may be pre-treated with the pH reactive film.

In still another example, the substrate (200) may be made of or may incorporate a material such as a paper that is colored white, for example, to contrast the reagent (105) with the substrate (200). In this example, if the reagent (105) has a color, then the reagent (105) will be visible in contrast to the material applied to the substrate (200). In another example, the material applied to the substrate (200) may be pH film or a fluorescent film that show when the reagent (105) is applied to the material. Instill another example, the pretreatment may include any combination of etching of the substrate (200), treating the substrate (200) with a pH film, and incorporation of the material such as a paper. Etching may include chemical or physical etching of the substrate (200).

In one example, the processor (414) may, with the reagent dispensing devices (104), deposit the reagent (105) on at least one of the diagnostic regions (202-1, 202-2) in a pattern. Because the reagent dispensing devices (104) are able to dispense the reagents (105) with great precision, and are, in some cases digitally addressable, the reagents (105) may be deposited on the substrate (200) with great precision and in a pattern that conveys information. The design of dispensed reagent (105) as deposited on the substrate (200) may be tailored to what kinds of failures that particular reagent (105) is vulnerable to. Further, the pattern of deposition of the reagent (105) on the substrate (200) may also be used as an additional way of identify what reagent (105) was dispensed on the substrate (200) in terms of a barcode that indicates a number of properties of the reagents (105) dispensed on the substrate (200) such as, for example, the type of reagent, a dispensed volume, a temperature at the time of dispensing the reagent (105), a date and time of the deposition of the reagent (105) or the test date, other information, or combinations thereof. The barcode may also be used to mark a substrate (200) as faulty. In this example, the substrate (200) may be marked with either the reagent (105) itself or using a reagent dispensing device (104) to indicate that something happened to compromise the reliability of the reagent (105) that was dispensed on that particular substrate (200). This would allow a technician to be made aware of the defective deposition of the reagent and possibly remove the defective substrate (200) from a test run.

In one example, the dispense patterns of the reagent (105) may include, for example, a pattern that indicates the health of the reagent dispensing devices (104) or parts of the reagent dispensing devices (104) such as, for example, nozzles from which the reagents (105) are dispensed. In this example, a number of diagnostics of stair step patterns may be deposited on the substrate (200) using the reagent dispensing devices (104) to indicate or demonstrate the health of each individual nozzle.

In another example, a decap pattern may be deposited by the reagent dispensing devices (104) onto the substrate (200). If the reagent (105) is known to, for example, clog nozzles from which the reagents (105) are dispensed, or has a tendency to decap within the reagent dispensing device (104), depositing the decap pattern on the substrate (200) may indicate the issue before the reagent (105) is deposited on the sample region (201) of the substrate (200).

In still another example, a concentration gradient may be deposited by the reagent dispensing devices (104) onto the substrate (200). The concentration gradient may include a number of areas of the diagnostic regions (202-1, 202-2) of the substrate (200) on which different volumes of a reagent are dispensed to obtain different concentrations along a length of the diagnostic regions (202-1, 202-2) or, in another example, a gradient of volumes across a length of the diagnostic regions (202-1, 202-2) of the substrate (200). The volume and concentration gradients may be used to calibrate the dispensed reagents (105) on the fly before the reagents (105) are dispensed on the sample region (201).

In still another example, the pattern of dispensed reagents (105) may include a barcode. The barcode may be any optical or imagable, machine-readable, representation of data. The reagent (105) may be deposited on at least one of the diagnostic regions (202-1, 202-2) of the substrate (200) as both a test to verify that the reagent (105) was deposited in a correct and effective manner, and as an identification of the diagnostic test performed on the substrate (200), the types of reagents (105) deposited on the sample region (201) of the substrate (200), the date and time the diagnostic test was conducted, other information specific to the substrate (200) and/or the diagnostic test, or combinations thereof. In this example, the optical sensors (140) may be used to capture an image of the barcode, and the processor (414) may cause the barcode to be read and understood for what information it conveys. In one example, the patterns of the reagents (105) may also identify a health of the reagent dispensing devices (104), identify a clogging of a number of nozzles of the reagent dispensing devices (104), a volume gradient of the reagent, a concentration gradient of the reagent, information specific to the substrate (200) and/or the diagnostic test, or combinations thereof.

The processor (414) may also, in response to a verification of the deposition of the reagent (105) on at least one of the diagnostic regions (202-1, 202-2) of the substrate (200), service the reagent dispensing devices (104). Once a diagnostic pattern has been deposited on at least one of the diagnostic regions (202-1, 202-2) of the substrate (200), a variety of responses may be included in the processes of the reagent dispensing system (400). The pattern of reagents (105) may be visually checked by a technician after the substrates (200) are collected, or the optical sensors (140) may be used to image the dispensed reagents (105) as they pass through the reagent dispensing system (400). In one example, a potential response to such an imaging of the substrates (200) may include servicing the reagent dispensing devices (104) within the reagent module (103). If the pattern indicates that a issue exists with the reagent dispensing devices (104) and their ability to properly dispense the reagents (105), then a separate region of the substrate (200) such as an unused portion of the diagnostic regions (202-1, 202-2) may be used for spitting reagent (105) to clear the reagent dispensing devices (104). A “spitting” routine, is an action taken by the processor (414) instructing at least one of the reagent dispensing devices (104) to purge its reagent ejection elements such as its nozzles by sending it a sequence of fire pulses, possibly of greater energy than the normal firing pulse. This serves to ensure that the reagent (105) contained in the nozzles does not dry, causing a blockage of dry reagent, which stops the nozzle from firing correctly. Spitting routines also help to clear already blocked, or partially blocked nozzles, which may be caused by fibers or dried reagent, for example.

Servicing the reagent dispensing devices (104) in response to a verification of the deposition of the reagent (105) may also include a wiping process. The wiping process may be achieved by wiping off or cleaning the reagent dispensing devices (104) of the reagent module (103) using the wiping station (403). The wiping process may be executed before the reagent is deposited by the reagent dispensing devices (104) in order to allow the reagent dispensing devices (104) to properly dispense the reagent (105).

Servicing the reagent dispensing devices (104) in response to a verification of the deposition of the reagent (105) may also include changing a nozzle mask of the reagent dispensing devices (104). In this example, if the diagnostic pattern deposited on the diagnostic regions (202-1, 202-2) of the substrate (200) was a volume calibration, this deposition of the volume calibration may be followed by a change in a nozzle mask of at least one of the reagent dispensing devices (104) to correct for the volumes of the reagent dispensed.

Servicing the reagent dispensing devices (104) in response to a verification of the deposition of the reagent (105) may also include changing a nozzle mask of the reagent dispensing devices (104). In this example, if the diagnostic pattern deposited on the diagnostic regions (202-1, 202-2) of the substrate (200) was a concentration calibration, this deposition of the concentration calibration may be followed by a change in a nozzle mask of at least one of the reagent dispensing devices (104) to correct for the concentration of the reagent.

In another example, servicing the reagent dispensing devices (104) in response to a verification of the deposition of the reagent (105) may include marking a substrate (200) as faulty. In one example, the substrate (200) may be marked with either the reagent (105) itself or using a reagent dispensing device (104) to indicate to the reagent dispensing system (400) and/or a technician operating the reagent dispensing system (400) that something happened to compromise the reliability of the reagent (105) that was dispensed on that particular substrate (200). This would allow the technician to be made aware of the defective deposition of the reagent and possibly remove the defective substrate (200) from a test run.

Aspects of the present system and method are described herein with reference to flowchart illustrations and/or block diagrams of methods, apparatus (systems) and computer program products according to examples of the principles described herein. Each block of the flowchart illustrations and block diagrams, and combinations of blocks in the flowchart illustrations and block diagrams, may be implemented by computer usable program code. The computer usable program code may be provided to a processor of a general-purpose computer, special purpose computer, or other programmable data processing apparatus to produce a machine, such that the computer usable program code, when executed via, for example, the processor (414) of the reagent dispensing system (100, 400) or other programmable data processing apparatus, implement the functions or acts specified in the flowchart and/or block diagram block or blocks. In one example, the computer usable program code may be embodied within a computer readable storage medium; the computer readable storage medium being part of the computer program product. In one example, the computer readable storage medium is a non-transitory computer readable medium.

The specification and figures describe a reagent substrate that may include a reagent sample deposition area on which a sample of a reagent is deposited, and a number of diagnostic regions on which a diagnostic sample of the reagent is deposited for verification of deposition of the diagnostic sample. Further, a reagent dispensing system may include a conveyor surface to convey a number of substrates, at least one reagent module located in-line with respect to the conveyor surface where the reagent module includes at least one reagent dispensing device to dispense a reagent on the substrates, and at least one optical sensor to verify the dispensing of the reagent at a number of diagnostic regions of the substrate.

The systems and methods described herein include a number of reagent dispensing devices (104) that provide for faster and more accurate treating of substrates within a reagent dispensing system than hand-pipetted methods. Further, these systems and methods also use less reagent and may provide more flexibility and complexity to the design of an experiment such as a diagnostic test run in terms of using multiple reagents on a single substrate. This method of dispensing reagents may not be useful unless there was a method and system of verifying that the desired pattern and amount of reagent was deposited on the sample. By performing the verification methods described herein, the substrate, in line with the actual dispensing of the reagent, makes it possible to ensure every substrate is treated as intended. These methods and systems also help maintain a regular servicing schedule for the reagent dispensing devices and ensure that the diagnostic testing performed is done so in an expeditious and effective manner.

The preceding description has been presented to illustrate and describe examples of the principles described. This description is not intended to be exhaustive or to limit these principles to any precise form disclosed. Many modifications and variations are possible in light of the above teaching.

What is claimed is:

1. A computer program product for on-substrate verification of reagent deposition, the computer program product comprising:
a computer readable storage medium comprising computer usable program code embodied therewith, the computer usable program code to, when executed by a processor:
identifying a sample region of a substrate;
identifying at least one diagnostic region of the substrate;
with a reagent dispensing device, depositing a reagent on the at least one diagnostic region; and
verifying deposition of the reagent at the at least one diagnostic region of the substrate.

2. The computer program product of claim 1, comprising computer usable program code to, when executed by a processor, instruct at least one optical sensor to verify the deposition of the reagent at the at least one diagnostic region.

3. The computer program product of claim 1, comprising computer usable program code to, when executed by a processor, pretreat the substrate, the pretreating facilitating the verification of the deposition of the reagent at the diagnostic region.

4. The computer program product of claim 1, wherein identifying at least one diagnostic region of the substrate comprises identifying a first diagnostic region on the substrate before a sample deposition area with respect to a direction of travel of the substrate, identifying a second diagnostic region on the substrate after a sample deposition area with respect to the direction of travel of the substrate, or combinations thereof.

5. The computer program product of claim 3, wherein pretreating the substrate comprises etching the at least one diagnostic region, pH treating the at least one diagnostic region, applying a label to the at least one diagnostic region, or combinations thereof.

6. The computer program product of claim 1, comprising computer usable program code to, when executed by a processor, with a reagent dispensing devices, depositing the reagent on the at least one diagnostic region in a pattern.

7. The computer program product of claim 6, wherein the pattern identifies a health of the reagent dispensing devices, identifies a clogging of a number of nozzles of the reagent dispensing devices, comprises a volume gradient of the reagent, comprises a concentration gradient of the reagent, comprises a barcode identifying the substrate, comprises a barcode identifying a sample test protocol, or combinations thereof.

8. The computer program product of claim 1, comprising computer usable program code to, when executed by a processor, and in response to the verification of the deposition of the reagent at the at least one diagnostic region of the substrate, service the reagent dispensing devices.

9. The computer program product of claim 1, comprising computer usable program code to, when executed by a processor, change a nozzle mask in response to a determination that a volume of the reagent deposit on the at least one diagnostic region is incorrect.

10. The computer program product of claim 1, comprising computer usable program code to, when executed by a processor, mark the substrate as faulty in response to a determination that the deposition of the reagent was compromised.

11. A reagent dispensing system comprising:
a conveyor surface to convey a number of substrates:
at least one reagent module located in-line with respect to the conveyor surface, the reagent module comprising at least one reagent dispensing device to dispense a reagent on the substrates; and
at least one optical sensor to verify dispensing of the reagent at a number of diagnostic regions of the substrate.

12. The reagent dispensing system of claim 11, wherein the reagent dispensing device is a digitally addressable fluid ejection device.

13. The reagent dispensing system of claim 11, wherein the at least one optical sensor comprises two optical sensors, the two optical sensors comprising:
a first optical sensor to image the substrate previous to the dispensing of the reagent on the substrate; and
a second optical sensor to verify dispensing of the reagent after a dispensing operation performed by the reagent module.

14. A reagent substrate comprising:
a reagent sample deposition area on which a sample of a reagent is deposited; and
a number of diagnostic regions on which a diagnostic sample of the reagent is deposited for verification of deposition of the diagnostic sample.

15. The reagent substrate of claim 14, wherein the diagnostic regions of the substrate comprise untreated portions, etched portions, pH treated portions, labeled portions, or combinations thereof.
